(19) United States
(12) Patent Application Publication
(10) Pub. No.: US 2016/0353709 A1
(43) Pub. Date: Dec. 8, 2016

(54) DEVICE FOR DETERMINING MOVEMENTS OF AN ANIMAL

(71) Applicant: N.V. NEDERLANDSCHE APPARATENFABRIEK NEDAP, Groenlo (NL)

(72) Inventors: Jeroen Martin VAN DIJK, Enschede (NL); Egbert Gert Jan UNINGE, Twello (NL); Woltherus KARSIJNS, Nijbroek (NL)

(73) Assignee: N.V. NEDERLANDSCHE APPARATENFABRIEK NEDAP, Groenlo (NL)

(21) Appl. No.: 15/058,811

(22) Filed: Mar. 2, 2016

Related U.S. Application Data
(63) Continuation of application No. 13/386,703, filed on Apr. 4, 2012, now abandoned.

(30) Foreign Application Priority Data
Jul. 24, 2009 (NL) 2003276

(51) Int. Cl. A01K 11/00; A01K 29/00; G01P 1/12; A61B 5/00; A61D 17/00; A01K 27/00; A61B 5/11

(52) U.S. Cl. CPC A01K 11/006; A01K 11/004; A01K 27/009; A01K 27/001; A01K 29/005; A61B 5/1123; A61B 5/6828; A61B 5/6822; A61B 5/6815; A61D 17/002; A61B 5/0002; A61B 5/4866; A61B 5/1116; A61B 5/1118; G01P 1/127; A61B 2503/40

(57) ABSTRACT

The device for determining movements of an animal comprises a housing, at least one G-sensor for detecting movements of the housing and for generating signals representing the detected movements. The device further comprises an electronic circuit connected to the G-sensor for processing signals received from the G-sensor so as to obtain processing results, transmitting means connected to the electronic circuit for transmitting the processing results and a power supply for at least feeding energy to the G-sensor and the electronic circuit wherein the G-sensor, the electronic circuit, the transmitter means and the power supply are located within the housing. The electronic circuit may be arranged for switching the G-sensor on and off according to a predetermined time pattern so as to save energy.

The invention relates to a device for determining movements of an animal.

Such a device is disclosed in for example, Dutch patent 1012872. This known device is provided with a position switch which is connected with an electronic counting circuit and a power supply. The position switch is arranged to switch in response to a relevant movement of the animal The number of times that the position switch switches is counted by means of the counting circuit. In this manner the device is arranged for determining relevant rest times of the animal.

A disadvantage of the known device is that the counting switch comprises moveable parts which are subject to wear.

The object of the invention is to provide a solution for at least the above referred to problem.

According to the invention, the device comprises a housing, at least one G-sensor for detecting movements of the housing and for generating signals representing the detected movements, an electronic circuit connected to the G-sensor for processing signals received from the G-sensor so as to obtain processing results, transmitting means connected to the electronic circuit for transmitting the processing results and/or information representing the generated signals and a power supply for at least feeding energy to the G-sensor and the electronic circuit wherein the G-sensor, the electronic circuit, the transmitter means and the power supply are located within the housing. The advantage of the G-sensor is that it comprises no moveable parts which are subject to wear.

Another disadvantage of the known device is that the position switch is only sensitive in a predetermined direction for movements of the animal. This means that only a limited number of different kinds of movements can be detected such as in this case lying down of the animal. According to a preferred embodiment the G-sensor is sensitive for accelerations in each possible direction so that the device can be used for several purposes such as counting the steps of an animal, registering predetermined movements such as detecting movements of the head of an animal, detecting the lying or laying down of the animal, detecting standing still of the animal, detecting of walking of the animal etc.

According to a preferred embodiment according to the invention, the device is characterized in that the electronic circuit is arranged for switching the G-sensor on and off according to a predetermined time pattern so as to save energy. This embodiment has the advantage that the lifetime of the power supply can be extended. Because a G-sensor may use relatively more energy than the use of, for example, a position switch, it is extremely advantageous to switch the G-sensor on and off according to a predetermined time pattern so as to save energy. Preferably the housing of the device is sealed against fluids and gasses. Moreover, preferably the housing cannot be opened without destruction of the housing. This means that the power supply cannot be changed for a fresh power supply. The power supply can be for example, a lithion-ion power supply. It may well be possible that the power supply can be used for a period of six to eight years if the G-sensor is switched on and off according to a predetermined time pattern. A further advantage is that the volume of the battery can now be limited if desired because a lower averaged power is required by the sensor. If the volume of the battery is decreased, the volume of the housing of the sensor may also decrease. Such a decreased volume may be beneficial because, in use, there is less chance that the housing may be damaged and also it is more convenient for the animal if the housing has a smaller volume.

According to a very sophisticated embodiment, the device is characterized in that the device is arranged to detect a certain predetermined movement or predetermined orientation of the device by means of the G-sensor wherein the electronic circuit is arranged to switch off the G-sensor for Q seconds if said predetermined movement or orientation is detected wherein, for example, Q is in the range of 1-1200, preferably 3-600, more preferably 6-60. For example, the predetermined movement or orientation of the device may correspond with the movement or orientation which the device will make or have, in use when attached to the animal, if the animal lies or lays down. More specifically if the animal lays down the animal makes specific known movements which preferably can be detected and recognised by the device if the device is attached to the animal (for example to a leg, tail, head, etc. of the animal). If the animal lies the animal also makes specific known movements which preferably can be detected and recognised by the device if the device is attached to the animal (for example to a leg, tail, head, etc. of the animal). The device may also be arranged to detect by means the G-sensor a specific orientation of the device if, in use, the device is attached to the animal (such as the animal's leg, tail, head, etc.). For example while the animal lies a specific orientation of a part of the animal, which orientation specifically occurs if the animal lies, can be detected and recognised by means of the device if the device is attached to this part of the animal. Also if the animal lays down a specific orientation of a part of the animal, which orientation specifically occurs if the animal lays down, can be detected and recognised by means of the device if the device is attached to this part of the animal. These orientations can for example be determined by means of the G-sensor on the bases of the integration of detected movements of the animal by means of the G-sensor and with reference to a known initial orientation of the device.

This embodiment is based on the insight that, if the animal lies or lays down, it will usually lie for a relatively longer time; say more than five minutes. Based on this knowledge, it is no longer useful to activate the G-sensor for measuring the movements of the animal during a time period that the animal is probably lying. This would be a waste of energy. In order to save energy, the G-sensor is switched off for, for example, two minutes. After having been switched off, the electronic circuit will again switch on and off the G-sensor according to the predetermined time pattern for detecting movements of the animal.

Preferably, the transmitting means comprise a responder provided with a resonant circuit arranged to respond by transmission of the processing results if the resonant circuit is present within an electromagnetic field having a frequency component corresponding with a resonant frequency of the resonant circuit. Other options are however also possible. For example the transmitting means may be arranged to communicate to a wireless network based on for example wifi, bluetooth etc. The data transmitted by the transmitting means may be send to a (central) external computer for further evaluating the processing results, for example for determining animal behaviour, recognising irregular animal behaviour, statitical analyses etc. Also the data transmitted may comprise information about the generated signals. This information may correspond with the generated signals as such and basically represent the raw information Obtained by means of the G-sensor for further processing by means of a external (central) computer. The main task of the electronic circuit may in that case be to provide processing results based on which the G-sensor is switched on and off respectively so as the save energy. Also preferably, the housing is sealed against fluids and gasses wherein preferably the housing can not be opened without destruction of the housing. According to a special embodiment the device is arranged to be attached to an animals' leg. It is however also possible that the device is arranged to be attached for example, to an animals' neck. A possible embodiment of the device according to the invention will now be described by means of the drawing wherein:

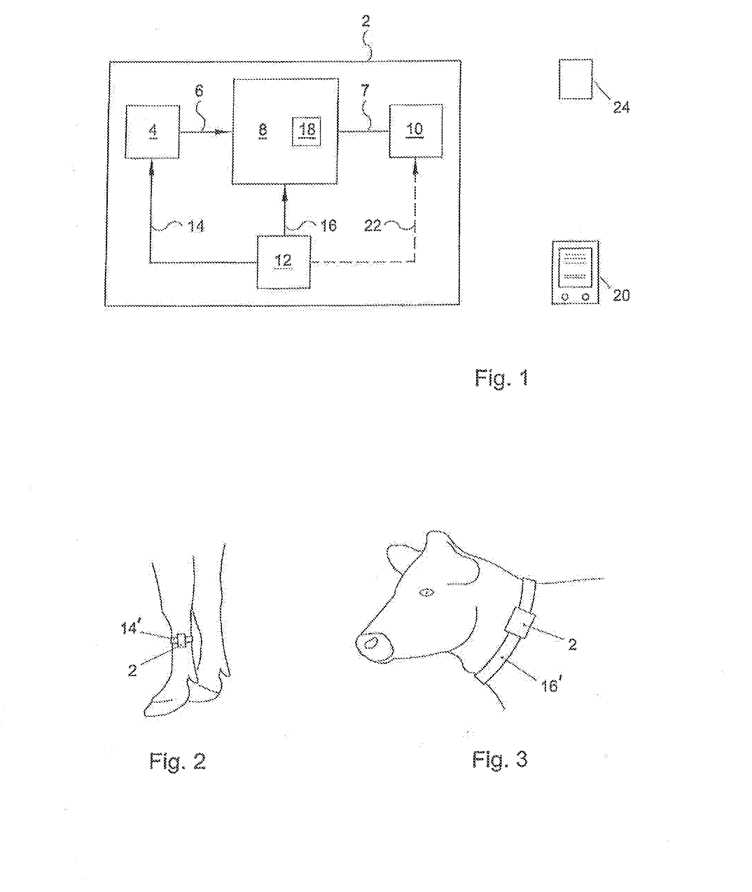

FIG. 1 discloses a schematic view of a possible embodiment of a device according to the invention;

FIG. 2 shows the device when it is arranged to be attached to an animals' leg; and

FIG. 3 shows the device when it is arranged to be attached to an animals' neck.

In FIG. 1 a device for determining movements of an animal is denoted with reference number 1. The device 1 comprises a housing 2. Furthermore the device is provided with a G-sensor 4 for detecting movements of the housing. The G-sensor is a well known G-sensor which is arranged for generating signals on a lead 6 which signals represent the detected movements of the G-sensor. The G-sensor is fixed within the housing so that the movement of the housing 2 corresponds with the movement of the G-sensor 4. The G-sensor is arranged for detecting accelerations of the G-sensor as well as the direction of said accelerations. In this example accelerations also include accelerations with a negative sign meaning de-accelerations. Moreover in this embodiment the G-sensor is arranged to detect accelerations in each possible direction.

The device further comprises an electronic circuit 8 connected to the G-sensor by means of the lead 6. The electronic circuit is arranged for processing signals received from the G-sensor so as to obtain processing result. The device further comprises transmitting means 10 connected to the electronic circuit 8 by means of a lead 7 for transmitting the processing results. Furthermore the device comprises a power supply 12 for feeding energy to at least the G-sensor and the electronic circuit via leads 14 and 16 respectively. The power supply is in this example a battery such as a nickel lithium battery which cannot be recharged. The G-sensor 4, the electronic circuit 8, the transmitting means 10 and the power supply 12 are located within the housing 2.

If the device moves, said movements are detected by the G-sensor. The G-sensor generates signals representing the detected movements. The electronic circuit receives the generated signal and processes said signals for obtaining processing results. If the device is arranged to be a device for counting steps of an animal, the processing results may be the number of steps which are detected within at least one time period. It may for example be that the day is divided in 24 time periods of one hour and that for each time period the number of steps is detected. It may also be that the processing results are the number of steps an animal has taken per time period. The time period may be, for example, one minute. For detecting such steps the device may be arranged to be attached to an animals' leg as shown in FIG. 2. In FIG. 2 the device 2 is provided with a strap 14′ for attaching the device to the animals' leg.

The processing results may also be of another nature. It may for example be that the device is arranged to be attached to the animals' neck as shown in FIG. 3. In FIG. 3 the device 1 is provided with a strap 16′ for attaching the device to the animals' neck. The processing results may for example be a detection that the animal has made a predetermined movement such as an estrous movement of the head. It may for example be that for that purpose the device is arranged to be attached to the animals neck as shown in FIG. 3. In FIG. 3 the device 1 is provided with a strap 16′ for attaching the device to the animals' neck, it may also be that the processing results are a detection that the animal is eating or ruminating. It may also be that the processing results are a movement of the animal, more particularly a movement of a leg of the animal if the animal is lying or laying down in that case the device may for example be attached to the animals' leg.

It is also possible that the processing results are the length of time the animal has walked and/or the length of time the animal has lied and/or the length of time an animal has been standing still within certain time period. For this purpose the device may be attached to the animals' leg. The processing results may also be the length of time an animal has walked and/or the length of time the animal has lied and/or the length of time an animal has been standing still per time period. In the latter case the device may be arranged as indicated in FIG. 2.

The electronic circuit 8 is in this example provided with a memory 18 for storing the processing results. The transmitting means 10 may comprise a well known responder provided with a resonant circuit which responds by transmission of the processing results which are stored in a memory 18 if the responder is brought within an electromagnetic field which is transmitted by a reader 20. The electromagnetic field has a frequency component corresponding with a resonance frequency of the resonant circuit of the responder. The responder obtains its energy from the electromagnetic field transmitted by the reader 20. Basically the responder modulates the electromagnetic field on the basis of the processing results which are stored in the memory 18. Therefore, in this example, the transmitting means is not powered by the power supply 12. The device may be arranged such that if the processing results of the memory 18 are read out by means of the reader 20, subsequently the processing results in the memory 18 are erased so that fresh memory locations are created for storing new processing results of a kind as explained above.

The G-sensor may be of a type which requires 1.2 Volt on lead 14 for its operation. In use, it uses a current in the order of micro Amperes so that it uses a power in the order of microwatts. The same applies to the electronic circuit 8. Although this does not seem to be much, this power consumption will provide only a limited life time for the power supply 12 and thereby for the device 2 because in this example the device 2 is sealed against fluids and gasses wherein the housing cannot be opened without destruction of the housing.

In order to extend the lifetime of the device, the electronic circuit 8 is arranged for switching the G-sensor on and off according to a predetermined time pattern so as to save energy. The time pattern according to which the electronic circuit is arranged for switching the G-sensor on and off is permanently stored in the memory 18. During the time the G-sensor is turned off, the G-sensor does not consume power so that energy is saved. There are several embodiments possible for the predetermined time pattern. A limited number of examples will be provided hereinafter. The invention is not limited to these examples. The electronic circuit may for example be arranged for switching the G-sensor on and off n times per minute wherein n is in the range of 10-3600, preferable within a range of 30-1200, more preferably within the range of 80-600. If for example the G-sensor is switched on and off 60 times per minute, this means that the G-sensor is switched on and off every second. Depending on the length of time the G-sensor is switched off every second, more or less energy can be saved. The electronic circuit may for example be arranged for switching the G-sensor on and off such that the G-sensor is on m % of time and turned off (100−m) % of time wherein m is in the range of 0.01-50, preferable within the range of 0.05-10, more preferably within the range of 0.01-5. Preferably the electronic circuit is arranged for keeping activated the G-sensor for substantially the same length in time each time the G-sensor is turned on.

It may also be that the electronic circuit is arranged for keeping de-activated the C-sensor for substantially the same length in time, each time the C-sensor is turned off.

It is also possible that the electronic circuit is arranged for keeping the G-sensor active for T seconds if the G-sensor is switched on wherein T is in a range of 0.001-10, preferably within the range of 0.001-1, more preferably within the range of 0.01-0.1.

According to a very special embodiment the device is arranged to detect a certain predetermined movement of the device by means of the G-sensor wherein the electronic circuit is arranged to switch off the G-sensor for Q seconds if said predetermined movement is detected. Q may for example be in the range of 1-1200, preferably 3-600, more preferably 6-60. The predetermined movement may for example be a movement from the animal. If it lies or a movement from the animal if it lays down. In those cases it is known that an animal generally will lie for a relatively long period of time, for example for more than one minute, so that the G-sensor can be switched off for, for example, 30 seconds. In that case there is little chance that a subsequent movement of the animal will not be detected because there is a relatively high chance that the animal will for more than 60 seconds. Instead of detecting a certain predetermined movement, it is also possible that the device is arranged to detect a predetermined orientation of the device by means of the G-sensor wherein the electronic circuit is arranged to switch off the G-sensor for, for example, Q seconds if said predetermined orientation is detected wherein for example Q is in the range of 1-1200, preferably 3-600, more preferably 6-60. The predetermined orientation may for example be an orientation of (a part of) the leg of the animal relative to the floor if the animal lies or if the animal lays down. For example the longitudinal lower part of the leg makes an angle of 45 degrees relative to the floor if the animal lies. Such orientation is a strong indication that the animal is lying or has lied. The device may be arranged in a well known manner to detect such an orientation by means of the G-sensor. For example the device may be attached to an animals leg. If the animal walks the device may be arranged to detect and recognise these walking movements by means of the G-sensor. If the animal walks, as a starting point a vertical orientation of the leg and therby of the device can be assumed by the device. Based on this orientation as a starting orientation new orientations can be calculated by the device based on the movements detected by means of the G-sensor. For example by integrating the detected movements a new orientations relative to the starting orientation can be calculated and thereby detected by the device.

In this example it holds that after the G-sensor is switched off for said Q seconds, the electronic circuit will start switching on and off again according to the predetermined time pattern so as to save energy as explained above. This means that there are basically in this example two ways of saving energy. The first way is switching off the G-sensor for a relatively longer period in time if the animal is lying. The other one is switching the G-sensor on and off according to the predetermined time pattern wherein the predetermined time pattern is selected such that no or relatively little information about the animals moving behaviour is missed.

In this example the processing results are recorded within the memory 18 per block of time wherein a block has a length of P seconds wherein P is in the range of 1-86400, preferably within the range of 60-14400, more preferably within the range of 90-7200. The device may be arranged to transmit the processing results per block of time. A block of time may for example be a block of four hours so that each day is divided into six of said blocks. The processing results may be as discussed above, for example, the length of time an animal has walked and/or the length of time the animal has lied and/or the length of time the animal has been standing still within a certain time period. The processing results may also be how often an animal lies down. How often an animal lies down may be determined in the number of times the animal lays down, for example, per hour.

As explained, a time period may be a period of one hour if the day is divided into 24 of such time periods. In such an example the processing results of four of such time periods is recorded into one block of time. Each block of time provides four time periods and six of such time blocks cover one full day. In such an embodiment, the start of a block of time corresponds with the start of one of the predetermined time period. It is of course also possible that each time period corresponds with one block of time.

It is also possible that the processing result is for example the length of time an animal has walked and/or the length of time the animal has lied, and/or the length of time an animal has been standing still per time period. As explained the time period may for example be one minute. Within one hour for example ten of such values may be determined. In such a case the processing results for fourty of such values is stored in one block of time if for example one block of time is four hours as in the example above. It is of course possible that a block of time has also another length such as one hour, thirty minutes etc. The frequency according to which said values are obtained may also differ from fourty.

The invention is in no way limited to the above described embodiment. It may, for example be that the transmitting means comprises a transmitter which uses energy of the power supply wherein this energy is supplied to the transmitter by means of lead 22. In that case the device may be arranged such that the transmitter of the transmitting means is activated automatically by means of the electronic circuit for, for example, transmitting the processing results which are stored in a block of time. In case a block of time corresponds with four hours, it may, for example, be that the processing results are transmitted every four hours. In such a manner also only a limited amount of energy is used by the transmitting means from the power supply. The advantage is that transmitting need not to be in a vicinity of a reader 20. The only thing which is required in such a case is that the transmitted data can be received by a receiver 24 which may be positioned, for example, somewhere on a farm where the animal which carries the device is present. Such modifications are all understood to fall within the scope of the invention.

1-35. (canceled)

36. A device for determining movements of an animal wherein the device comprises a housing, at least one G-sensor for detecting movements of the housing and for generating signals representing the detected movement, an electronic circuit connected to the G-sensor for processing signals received from the G-sensor so as to obtain processing results, transmitting means connected to the electronic circuit for transmitting the processing results and/or information representing the generated signals and a power supply for at least feeding energy to the G-sensor and the electronic circuit wherein the G-sensor, the electronic circuit, the transmitter means and the power supply are located within the housing, characterized in that the electronic circuit is arranged for switching the G-sensor on and off according to a predetermined time pattern, wherein during the time that G-sensor is turned off, the G-sensor does not consume power so as to save energy; wherein the electronic circuit is arranged for keeping the G-sensor on for T seconds if the G-sensor is switched on wherein T is in the range of 0.001-10.

37. The device according to claim 36, characterized in that the electronic circuit is arranged for switching the G-sensor on and off a times per minute wherein n is in the range of 10-3600.

38. The device according to claim 36, characterized in that the electronic circuit is arranged for switching the G-sensor on and off such that the G-sensor is on m % of time and off for (100−m)% of time wherein m is in the range of 0.01-50.

39. The device according to claim 38, characterized in that the electronic circuit is arranged for keeping the G-sensor on for substantially the same length in time each time the G-sensor is switched on.

40. The device according to claim 38, characterized in that the electronic circuit is arranged for keeping the G-sensor off for substantially the same length in time each time the G-sensor is switched off.

41. The device according to claim 36, characterized in that the device is arranged to detect a certain predetermined movement or predetermined orientation of the device by means of the G-sensor wherein the electronic circuit is arranged to switch off the G-sensor for Q seconds if said predetermined movement or orientation is detected wherein Q is in the range of 1-1200.

42. The device according to claim 36, characterized in that after the G sensor is switched off for Q seconds the electronic circuit will start switching the G-sensor on and of again according to the predetermined time pattern so as to save energy.

43. The device according to claim 41, characterized in that, the predetermined movement of the device corresponds with a movement which the device will make, in use when attached to the animal, if the animal lies or lays down and/or that the predetermined orientation of the device corresponds with the orientation which the device will have, in use when attached to the animal, if the animal lies or lays down.

44. The device according to claim 36, characterized in that, the processing results are recorded within a memory of the electronic circuit per block of time wherein a block of time has a length of time P seconds wherein P is in the range of 1-86400.

45. The device according to claim 44, characterized in that the device is arranged to transmit the processing results per block of time.

46. The device according to claim 36, characterized in that the processing results are a detection that the animal has made a predetermined movement.

47. The device according to claim 36, characterized in that the processing results are the length of time an animal has walked and/or the length of time the animal has lied and/or the length of time an animal has been standing still within a certain time period and/or a detection that the animal has made a predetermined movement.

48. The device according to claim 36, characterized in that the processing results are the length of time an animal has walked and/or the length of time the animal has lied and/or the length of time an animal has been standing still per time period.

49. The device according to claim 36, characterized in that the processing results are the number of steps an animal has taken within at least one time period.

50. The device according to claim 36, characterized in that the processing results are the number of steps an animal has taken per time period and/or how often the animal lies down.

51. The device according to claim 36, characterized in that the housing is sealed against fluids and gasses wherein the housing cannot be opened without destruction of the housing.

52. The device according to claim 36, characterized in that, the transmitting means comprise a responder provided with resonant circuit arranged to respond by transmission of the processing results and/or the information representing the generated signals if the resonant circuit is present within an electromagnetic field having a frequency component corresponding with a resonant frequency of the resonant circuit.

53. The device according to claim 36 characterized in that, the G-sensor is sensitive for accelerations in each possible direction.

54. The device according to claim 36, characterized in that, the G-sensor is arranged to detect accelerations of the G-sensor as well as a direction of said accelerations.

55. The device according to claim 36, characterized in that, the G-sensor is fixed within the housing.

56. The device according to claim 36, characterized in that, the device is arranged to be attached to an animals' leg.

57. The device according to claim 36, characterized in that, the device is arranged to be attached to an animals' neck, and/or that the device is arranged to be attached to the animals' head and/or ear and/or that the device is arranged to be attached to the animals' tail.

58. The device according to claim 37, wherein n is in the range of 30-1200.

59. The device according to claim 58, wherein n is in within the range of 60-600.

60. The device according to claim 38, wherein m is in the range of 0.05-10.

61. The device according to claim 60, wherein m is in the range of 0.1-5.

62. The device according to claim 36, wherein T is in the range of 0.001-1.

63. The device according to claim 62, wherein T is in the range of 0.01-0.10.

64. The device according to claim 41, wherein Q is in the range of 3-600.

65. The device according to claim 64, wherein Q is in the range of 6-60.

66. The device according to claim 44, wherein P is in the range of 60-14400.

67. The device according to claim 66, wherein P is in the range of 90-7200.

68. The device according to claim 46, wherein the predetermined movement is an estrous movement of the head for which the device is attached to the neck of an animal, a movement of the leg if the animal is lying or laying down for which the device is attached to a leg of the animal, a stepping movement of an animals' leg for which the device is attached to a leg of the animal.

69. The device according to claim 47, wherein the predetermined movement is an estrous movement of the head, a movement of the leg of the animal which is lying or laying down, a stepping movement of an animals' leg, and/or a movement of the animals' head if the animal is eating or ruminating.
